(19) United States
(12) Patent Application Publication
(10) Pub. No.: US 2004/0199223 A1
(43) Pub. Date: Oct. 7, 2004

(54) Laser system for treatment and diagnosis

(76) Inventors: Peter E. Andersen, Frederiksberg (DK); Paul Michael Petersen, Hillerod (DK); Eva Samsoe Andersen, Fredriksberg (DK); Sune Svanberg, Lund (SE); Katarina Svanberg, Lund (SE); Stefan Andersson-Engels, Lund (SE)

Correspondence Address: Ronald W Citowski, Gifford Krass Groh Sprinkle Anderson & Citkowski, Suite 400, 280 North Old Woodward Avenue, Birmingham, MI, 48009, US

(21) Appl. No.: 10/480,919

(22) PCT Filed: Jun. 14, 2002
(86) PCT No.: PCT/DK02/00405

(30) Foreign Application Priority Data
Jun. 15, 2001 (DK) PA 2001 00938

(51) Int. Cl. A61N 5/06

(52) U.S. Cl. 607/89

(57) ABSTRACT

The present invention relates to a system and a method for emitting plane laser light to biological tissue, wherein in high power lasers are modulated in order to provide an output beam consisting of high temporal and/or spatial coherence and at the same time is capable of delivering an output beam having a high power when being delivered through fibres having a core diameter of less than 400 μm. The method and system may in particular be used for photodynamic treatment (PDT) Optical fibers and/or thermal treatment. In a preferred embodiment the system and method also includes a monitoring system for the effect actually delivered to the biological tissue. Furthermore, the invention relates to a method of treatment using the system, as well as a method for diagnosing using the system.

The present invention relates to a system and a method for emitting laser light to a biological tissue, such as system and a method for treating and/or diagnosing conditions and/or diseases in an individual.

### BACKGROUND

For many medical purposes it is useful to apply a light, such as laser light to a biological tissue for treatment and/or diagnosis. The light is often supplied to the tissue by means of several fibres. For example, photodynamic therapy (PDT) is a promising treatment modality of malignant and pre-malignant tumors. PDT relies on the coexistence of three components: photosentisizer, light, and oxygen. The photosentisizer is administered to the patient, where it accumulates selectively in the desired tissue, such as cancerous cells. When the light excites the photosensitive drug a photochemical reaction occurs. This leads to the formation of singlet oxygen, which is a reactive and highly aggressive molecule. The singlet oxygen reacts with its surroundings, i.e. the diseased tissue, causing selective destruction of the cancerous cells. Using the photosentisizer δ-aminolevulinic acid (δ-ALA) requires light with a wavelength around 635 nm.

Traditionally the used light source for PDT treatment has been a Nd:YAG or an Arion laser pumped dye laser. However, these systems are inconvenient in a clinical environment due to their size, high cost and complex operation. Laser-induced thermo-therapy is most often based on NIR diode laser array systems.

The use of diode laser systems in medicine has increased rapidly during the past decade since their compactness, low cost, and simple operation make them attractive in a clinical environment. They are continuously replacing light sources previously used in PDT—large expensive systems that, in addition, are complex to operate. A remaining drawback of presently available high power diode laser systems is that they provide a low-quality laser beam. The high-power multimode diode laser sources, such as broad area lasers and laser bars are now available with output power up to 20 W or more, used for these treatments are small, low-cost and easy to operate, but they suffer from multimode non-diffraction limited output, which limits the coupling efficiency to optical fibres. This means that they have poor coupling efficiency to small-core diameter optical fibres and are only capable of delivering the treatment light through optical fibres with core diameters of 400 μm or more. Commercial diode lasers for medical laser treatments are available (e.g. from Diomed and CeramOptec), but these lasers are also only able to deliver the treatment light through fibres of a core diameter of 400 μm or thicker.

This limitation is especially important to PDT. For this application it can be difficult to find a laser with sufficient power through a thin fibre for optimal treatment. The optimal fibre core diameter in individual treatment cases is often less than 400 μm. In particular, when performing interstitial PDT treatments, It is desirable to have a thin treatment fibre. Thus, there is a need for a new light source delivering the therapeutic light through an optical fibre with a small core diameter.

Single-mode diode lasers provide spectrally and spatially coherent light with output power up to 200 mW. In many biomedical applications, including PDT, a higher out-put power is needed output cannot efficiently be coupled into single mode fibres or fibres with a small core diameter.

In WO 98/56087 a laser light system highly coherent, possibly single mode, output light beam is described, however no applications of this laser is described, and furthermore, there is no indication of the power obtained when using a single mode fibre.

In SE 9501278-7 a system for photodynamic therapy is described. The system provides the possibility of monitoring the treatment by using the light fibres as detectors as well. As light source a laser light is mentioned, without describing the light source further. The system calculates, from tissue optical parameters given, optimal source positions for treatment with a specified number of optical fibres. After placing the fibres in position, the light fluence predicted to destroy the lesion, while saving as much as possible of the surrounding tissue, is delivered.

Currently, there exists no suitable laser or light sources for the diagnostic procedure. Furthermore, there are no light source available for these applications capable of switching between pulsed and continuous mode.

### SUMMARY OF THE INVENTION

The present invention relates to a system and a method for emitting laser light to a biological tissue, whereby a high ratio of the laser effect is delivered through the fibre into the tissue, and preferably where the effect on the tissue may be monitored.

Accordingly, the present invention relates to a system for emitting laser light to a biological tissue comprising:

a laser light system capable of producing an output beam consisting of high temporal and/or high spatial coherence

focusing means for focusing said output beam into an at least substantially elliptic beam,

means for coupling said at least substantially elliptic beam into at least two delivery fibres.

In another embodiment the invention relates to a system for emitting laser light to a biological tissue comprising:

a laser light system having low spatial coherence,

means for improving the laser light system from low coherence to high coherence, to obtain a system capable of producing an output beam consisting of high temporal and/or high spatial coherence

focusing means for focusing said output beam, and

means for coupling said beam into at least one delivery fibre(s).

In a preferred embodiment the beam is finally coupled into at least two delivery fibres, such as at least three delivery fibres, such as at least four delivery fibres, such as at least five delivery fibres, such as at least six delivery fibres.

In yet another embodiment the invention relates to a system being a combination of the above described, wherein the laser system is improved and the astigmatism of the beam is corrected.

By the term “low spatial coherence” is meant that the degree of diffraction (M²) is larger than 10, such as larger than 20, such as larger than 30, up to about larger than 100.

By the term “high spatial coherence” is meant that a close to diffraction limited beam may be obtained from the laser system, whereby a large ratio of said beam may be delivered through the fibres to the tissue. By the term “a close to diffraction limited beam” is means a substantially diffraction limited beam, such as M²<2, such as M²<1.4. In particular for a single mode fibre it is preferred that M²<1.2. Preferably M² is between 1-10.

Thus, in a preferred embodiment the means for improving the spatial coherence of the light, preferably reduces M² by a factor in the range of from 5-100, such as from 5-50, such as from 5-25.

Accordingly, it is an object of the invention to use for example diode lasers capable of having the spatial coherence of the laser beam improved. However, although improved with respect to coherence it is still important that the improved beam also is able to deliver a sufficient power. Accordingly, it is preferred that the system according to the invention also is able to monitor the effect dosed to the tissue.

Furthermore, the invention relates to a method for emitting laser light to a biological tissue comprising:

arranging a laser light system capable of producing an output beam consisting of high temporal and/or spatial coherence,

focusing said output beam into an at least substantially elliptic beam,

coupling said at least substantially elliptic beam into at least two delivery fibre having a proximal end and a distal end, and

arranging the distal end of said delivery fibres in contact with the biological tissue, thereby emitting laser light to the biological tissue.

As well as to a method for emitting laser light to a biological tissue comprising:

arranging a laser light system having low spatial coherence,

improving the laser light system from low coherence to high coherence, to obtain a system capable of producing an output beam consisting of high temporal and/or high spatial coherence

focusing said output beam, and

coupling said beam into at least one delivery fibre(s).

The system and method according to the invention may be used for various treatment and diagnostic purposes. Accordingly, the invention relates to a system for treatment of a condition or disease, wherein said system being as defined above.

Furthermore, the invention relates to a method of treating a condition or disease relating to a tissue volume in an individual comprising,

identifying the tissue volume,

arranging delivery fibre(s) connected to a system as defined above or by the method as defined above, in contact with at least a part of said tissue volume,

emitting laser light through said delivery fibres to the tissue volume.

Also, the invention relates to a system for diagnosing a condition or disease, said system being as defined above as well as a method of diagnosing a condition or disease relating to a tissue volume in an individual comprising,

arranging at least two delivery fibres connected to a system as defined above or by the method as defined above, in contact with tissue suspected to comprise at least a part of said tissue volume,

emitting laser light through said delivery fibres to the tissue volume,

detecting a signal in at least one of the other delivery fibres, and

correlating said signal to the presence or absence of said condition or disease.

In yet a further aspect, the invention relates to a method for treating a condition or disease relating to a tissue volume in an individual and monitoring said treatment comprising

arranging at least two delivery fibres connected to a system as defined above or by the method as defined above, in contact with tissue suspected to comprise at least a part of said tissue volume,

emitting laser light through said delivery fibres to the tissue volume,

detecting a signal in at least one of the other delivery fibres, and

correlating said signal to the presence or absence of said condition or disease.

### DRAWINGS

FIG. 1: Shows a scheme of the mechanism of PDT. Laser light of approximately 635 nm excites the photosensitizer from its singlet ground state S₀ to the first excited singlet state S₁. Due to the small energy separation the photosensitizer molecules are able to relex to the lower, metastable, triplet state T₁ by intersystem crossing (IX). The excess energy is transferred to the surrounding molecules, most of which is oxygen, O₂, in its triplet ground state. The oxygen is thereby excited, resulting in the formation of singlet molecular oxygen. The very reactive and highly aggressive nature of singlet oxygen leads to the death of the surrounding diseased cells.

FIG. 2: Shows a side-view of the spatial filtering process. The spatial filter is adjusted to permit only one lobe (the injection lobe) to pass through and reach the external reflector. The other lobe (the output lobe) is significantly amplified and extracted from the system by the external reflector.

FIG. 3: Shows the laser system schematically.

FIG. 4: Shows an experimental setup viewed from above. L1 and L2 constitute a collimating lens pair. W is a wedge extracting two reflections to be used in beam diagnostics. E is an optional frequency selective element. MF denotes an example of spatial filtering and feedback in one plane. L3 and L4 are cylindrical lenses acting as a beam expander in the slow axis. L5 is a plane-convex lens, focusing the output into an optical fibre. This fibre is connected to a beam splitting, delivery and monitoring unit.

FIG. 5: Shows the intensity profile in the far field with (dashed) and without (freely running laser—full line) external feedback and spatial filtering. The distributions are shown as a function of the lateral angle, i.e. in the slow axis direction. The intensity distribution narrows down and assumes the double lobed shape, when the filtering and feedback are applied. The high intensity lobe centered at positive angle constitutes the output from the system.

FIG. 6: Shows a schematic view of the fibre-based delivery and monitoring system for multiple-fibres laser treatment of diseased tissue. 1: input coupler, 2-5: lenses, 3-4: beam splitters, 6: output couplers/detectors.

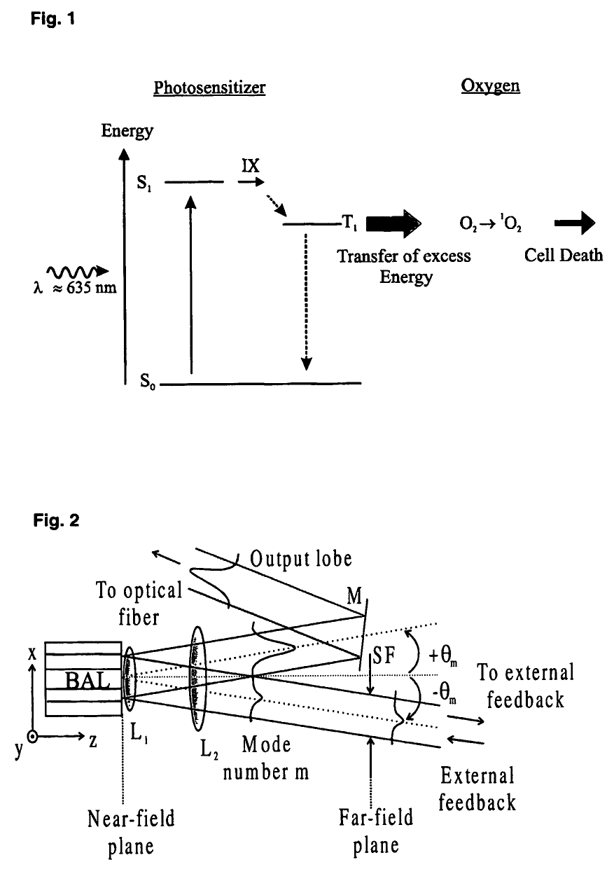

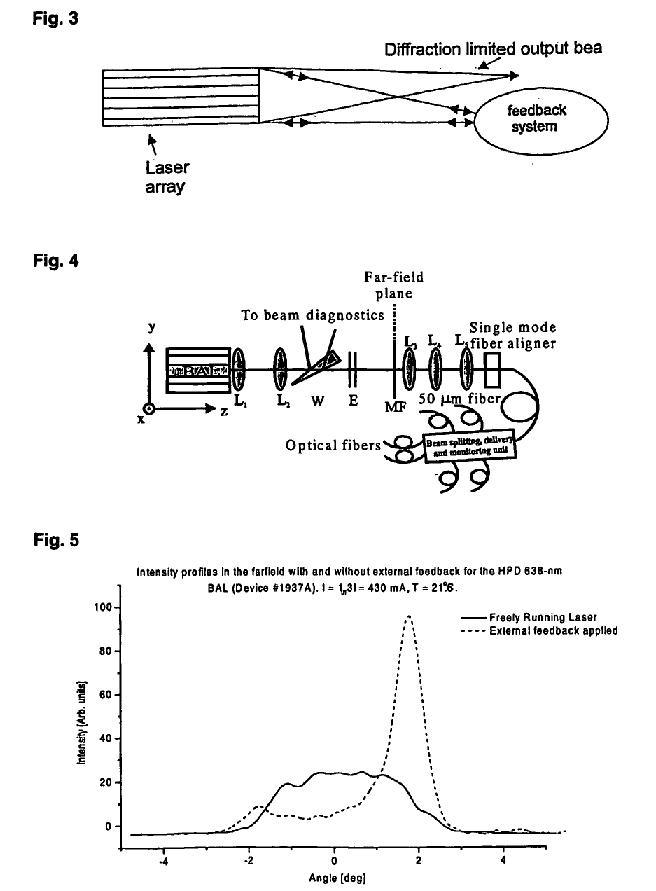

### DETAILED DESCRIPTION OF THE INVENTION

As discussed above the object of the present invention is to provide a system for emitting laser light to biological tissue, wherein a high ratio of the laser effect is actually delivered to the tissue in question. It has been found that in order to secure the large effect delivered to the tissue a high quality laser beam must be produced by the laser. Accordingly, it is a requirement that laser light system according to the invention is capable of producing an output beam consisting of high temporal and/or spatial coherence and at the same time is capable of delivering an output beam having a high power.

A high quality laser is in the present context preferably a laser having a high spatial coherence, and therefore in one aspect the invention relates to a improving the coherence properties of high energy laser to a diffraction degree (M²) of at most 10, such as of at most 5, such as of at most 2, such as of at most 1.4, such as of at most 1.2.

The laser may comprise any suitable laser beam generation means, such as a gas laser, a semiconductor laser, a semiconductor laser array, a superluminescent laser diode, a dye laser, a Nd-YAG laser, an Argon ion laser, etc.

Further, the laser may comprise any array of lasers of the above-mentioned types, such as a broad area laser or an array of broad area lasers, laser diode array, laser bar or stacked array, or a laser diode etc.

The invention is particularly useful for lasers having a broad bandwidth gain medium, such as dye lasers, semiconductor lasers, titanium sapphire lasers, F-center lasers etc.

A broad area laser is a linear array of high gain regions from which laser beams may be emitted and separated by low-gain regions. A broad area laser may provide a laser beam with an output power of up to 2 Watts. If a higher output power is desired, the cross section of the gain medium of the semiconductor laser may be increased and/or several laser elements may be combined into an array. Broad area lasers may themselves be combined into an array of broad area lasers, such as laser bars, providing a laser beam with an output power of 20 Watts.

In the present context, a laser is said to emit a high power output light beam when it is pumped with energy at a level substantially above the threshold level. The threshold level is the lowest possible energy level at which the laser can lase. It is well known that laser may be pumped with various types of energy, such as electrical energy, electromagnetic energy, such as light, etc. For example, a semiconductor laser may be pumped with electrical energy by supplying a current to the laser. The semiconductor laser lases when the current supplied to it is greater than or equal to the threshold current of the laser and the laser is emitting a high power output light beam when the current supplied to it is substantially larger than the threshold current, such as 1.5 times the threshold current.

In the present context, in particular with respect to emitting laser light to biological tissue for treatment purposes, the effect of the laser light is important. In general the higher the energy the shorter treatment time. It is preferred for interstitial PDT that at least 100 mW/delivery fibre is delivered, such as at least 150 mW/derlivery fibre, such as at least 200 mW/delivery fibre. In order to use the energy efficiently, it is preferred that at least 25% of the light beam is coupled into the fibres, such as at least 30% of the light beam, such as at least 40% of the light beam.

Typically, broad area lasers support multiple longitudinal and spatial modes, thus the system is dynamic and the mode structure is constantly changing. This is an important disadvantage of broad area lasers.

In order to improve the laser system and produce a beam of high quality the laser light system preferably comprises means for external feedback, such as a laser system comprising a first laser for emission of a first high power light beam and a means for external feedback for emission of a second light beam in response to light incident upon it and being positioned in relation to the first laser so that, during emission of the first light beam, the device is illuminated by a first part of the first light beam and the second light beam is injected into the first laser, the means for external feedback and the first laser defining an external cavity there between. The invention thus implies a method of generating a coherent light beam, comprising the steps of: operating a first laser to emit a first high power light beam, forming an external cavity between a means for external feedback and the first laser by illuminating the means for external feedback by a part of the first light beam thereby causing emission of a second light beam from the means for external feedback, and injection of the second light beam into the first laser.

The external feedback system may be implemented as, but not limited to, one of the following configurations:

grating based with spatial filter, grating, and lenses,

mirror based with a mirror, gratings and a spatial filter,

holographic element.

For further improving the beam quality it is preferred that the means for external feedback comprises a spatial filter positioned in the light path of the first part of the first light beam and preventing transmission of selected spatial modes towards the means for external feedback.

The spatial filter included allows only one or few spatial modes to interact with the external feedback system. The spatial filtering may be performed with any suitable means, such as two razor blades mounted on translation stages.

The highest brightness is obtained when both a spatial filter and a frequency filter are applied at the same time.

Predetermined wavelength range(s) of the second light beam may be selected by positioning a frequency selective element in the external cavity in the light path of the part of the first light beam illuminating the means for external feedback whereby the laser system is controlled to emit a stable and highly spatially and temporally coherent output light beam with a narrow band spectrum, such as means for external feedback comprising an etalon.

The frequency selective element is an element that either deflects, such as by reflection, refraction, scattering, diffraction, etc, light propagating along a first propagation axis and impinging upon the frequency selective element into light propagating along a second propagation axis forming an angle with the first propagation axis, the size of the angle being dependent upon the wavelength of the impinging light, or, transmits a selected part of the impinging light within a specific wavelength range while the remaining part of the impinging light is absorbed and/or deflected.

The frequency selective element may comprise an interference filter, an absorbance filter, such as a semiconductor doped glass, etc, an etalon, a prism, a grating, such as a diffractive optical element, such as a hologram, etc., preferably an etalon.

The operating characteristics of the frequency selective element either alone, such as for an etalon, or in combination with the means for external feedback, such as for a grating, determines the wavelength range of the second light beam that is injected into the first laser.

The frequency selective element is positioned in the external cavity in the light path of the part of the first light beam illuminating the means for external feedback and it is adapted to select a specific wavelength range of the part of the first light beam illuminating the means for external feedback. In response to the illuminating light, the means for external feedback emits the second light beam having a wavelength range corresponding to the selected wavelength range so that the frequency selective element in cooperation with the light feedback device selects the wavelength range of the second light beam without reducing the total power of the output beam significantly.

The etalon is a frequency filter and will only pass a limited number of frequencies that can interact with the means for external feedback. Single spatial mode operation can be achieved if the orientation of the etalon is adjusted so that the wavelength for peak transmission matches the lasing wavelength of a spatial mode with high gain. In this case, the bandwidth of the output light may be less than 0.03 nm.

It is an important advantage of the invention that selection of a small wavelength range of the light beam injected into the adaptive light feedback element causes the first laser to be stabilized so that a centre wavelength of the first light beam remains constant substantially independent of various operating parameters, such as operating temperature of the first laser, mechanical vibrations of the system, light modulation of the first laser, etc.

The placement of an etalon in the external cavity causes narrow band operation. The light beam with a narrow band spectrum is defined as an output light beam with an optical power spectrum in which the full width at half maximum (FWHM) of the best fit to the optical power spectrum of a Gaussian envelope is less than one longitudinal mode spacing of the solitary free running laser. The longitudinal mode spacing is given by c/(2nL) where c is the speed of light and nL is the optical path length of the laser cavity, where n is the refractive index and L is the length of the laser cavity. However, the farfield energy distribution is still far from the diffraction limit. For a 100-ptm wide BAL at 815 nm wavelength, the diffraction limit is 0.55 degree. The diffraction limit being defined as FWHM of the lowest order BAL mode in the farfield (intensity profile) and given by 1.189X/2xo, where X is the wavelength and xo is the half-width of the BAL. When the laser is freely running the FWHM angular width of the farfield energy distribution pattern is 4 degrees (7.3 times the diffraction limit)

For the various purposes of diagnostic, thermo-treatment and photodynamic treatment it is preferred that the laser is capable of producing a beam having a wavelength in the range of from 250 nm to 1600 nm, such as from 300 nm to 1400 nm, such as from 400 nm to 800 nm.

Also, the laser system may further comprise means for non-linear generation of other frequencies, such as an intracavity means or preferably an extracavity means. Examples hereof are a frequency doubler, optical parametric oscillator, etc., for frequency conversion of at least part of the incident light beam so that the wavelength of the coherent light beam is selected to a desired wavelength.

For example, the laser system may further comprise a frequency doubler for frequency doubling at least part of the incident light beam so that the wavelength of the coherent light beam is substantially equal to half the wavelength of the incident light beam. This method may lead to new frequency doubled light sources with wavelengths ranging from 1 nm-50 μm, such as from 100 nm-10 μm, such as from 100 nm-3 μm, preferably such as from 100 nm-500 nm, such as 300 nm-550 nm.

Throughout the present description, it is to be understood that the frequency doubler may be substituted by a crystal of an optical parametric oscillator for generation of any desired wavelength.

Inside the external cavity the intensity of the light beam may be high, and positioning of the frequency doubler within the external cavity provides a high power frequency doubled output.

In the external cavity the frequency doubler may be placed inside the frequency selective element, for example inside an etalon formed by reflecting surfaces, or the surfaces of the frequency doubler may be the frequency selective element itself.

The intensity of the beam inside a frequency selective element, such as inside an etalon, is enhanced with a factor of I/(1−r)², where r is the reflectivity of the reflecting surfaces, relative to the beam outside the etalon. The intensity inside an etalon with a reflectivity at each surface of 0.9 is thus amplified with a factor 100. The intensity of the frequency doubled light is proportional to square of the intensity of the light incident on the frequency doubler and typically only a few percent of the incident light is frequency doubled when the frequency doubler is positioned in the external cavity. By positioning the frequency doubler inside the etalon in the external cavity or by having the surfaces of the frequency doubler constituting the frequency selective element of the external cavity, the beam to be frequency doubled has a high intensity and is highly collimated whereby the efficiency of the frequency doubling is increased.

Due to the high intensity, the stability, and high spatially and temporally coherence of the beam inside the external cavity, the invention also provides for the first laser to be a laser emitting a narrow band output light beam, which output light beam is to be frequency doubled thereby increasing the usefulness of the laser system for medical purposes.

The generation of other frequencies may relate to other frequencies having a wavelength in the range of from 250 nm to 1600 nm, such as from 300 nm to 1400 nm, such as from 400 nm to 800 nm.

It is another advantage of the invention that a laser system emitting a light beam of high optical brightness is provided, the optical brightness of a source is defined as the energy per unit area, per unit time, per unit solid angle, or per unit frequency interval.

The wavelength of the output light beam may be adjusted by corresponding adjustment of the wavelength range transmitted or deflected by the frequency selective element. Thus, a laser system with an adjustable wavelength may be provided.

For example, a laser system with a first laser with a free running centre wavelength equal to 800 nm may be adjusted +/−3 nm.

For example, when the frequency selective element comprises a grating, the laser system may comprise frequency adjustment means for selection of the frequency of the output light beam, the frequency adjustment means being adapted to adjust the angular tilt of the grating in relation to a propagation axis of the light beam illuminating the means for external feedback. As the grating deflects light into propagation directions that depend on the wavelength of the light, the wavelength of light impinging on the means for external feedback depends on the angular tilt of the grating in relation to the feedback element. The adjustment of the angular tilt of the grating does not result in a continuous tuning of frequencies, but in discrete frequency steps between different longitudinal modes, each of which belongs to the same spatial mode.

The angular tilt of the grating may be controlled by a piezo element. Hence, the frequency can be automatically adjusted by the application of a voltage to the piezo element.

Further, for a fixed position of the grating the wavelength may be continuously adjusted by varying the temperature of the first laser. By changing the temperature less than 1°, the wavelength may be adjusted over a wavelength range corresponding to one longitudinal mode spacing.

The high quality beam thus produced of high temporal and/or spatial coherence is then focussed to a beam capable of being coupled into at least one fibre. In a preferred embodiment the output beam from the laser is directed towards the focusing means by means of reflecting means.

The focusing means comprises means for expanding the output beam in at least one direction to correspond to at least substantially to the geometry of the cross-section of the fibre(s), such as a substantially elliptic beam, preferably a substantially circular beam. The focusing means may thus comprise means for expanding the output beam at least 4 times in at least one direction, such as at least 6 times, such as at least 8 times. A beam expander made of two cylindrical lenses (for example one f=25 mm and one f=150 mm) expands the smallest dimension of the output beam.

The coupling means preferably comprises means having a numerical aperture adapted to the thickness of the core of the fibre(s) or the bundle of fibres in order to sufficiently coupling the highest possible amount of light into the fibre, thereby reducing loss of energy in the coupling phase. The coupling means therefore preferably comprises a lens, such as an achromat or a triplet. The advantageous coupling efficiency obtained by such a system is caused by the output beam of the system being near diffraction limited and by the high brightness of the output light beam.

The fibres used in accordance with the present invention may be arranged in any suitable configuration of which two principally different arrangements are to be discussed here:

Coupling of the laser beam into at least one optical fibre, said optical fibre being serially coupled to at least two delivery fibres through for example beam splitters.

Coupling of the laser beam directly into a bundle of delivery fibres.

The delivery fibre is arranged in contact with the biological tissue to be treated, and for some applications the fibres are inserted into the tissue. In particular in the latter case, the fibre is preferably as thin as possible, taken into account that the fibre must not break during handling. In the present context the dimensions of the fibre, relates to the fibre as such, and not to any coating surrounding the fibre, for example for reinforcement purposes. The diameter of the core of the delivery fibre is preferably in the range of from 5 μm to 500 μm, such as from 25 μm to 200 μm, such as from 40 μm to 150 μm. The fibre(s) all have a proximal end facing the incoming beam and a distal end in contact with the tissue to be treated or diagnosed.

The diameter of the core of the optical fibre is adjusted to the diameter of the delivery fibre, so that as little as possible of the light energy is lost in the couplings. At least two delivery fibres are arranged in the system, such as at least three delivery fibres, such as at least four delivery fibres, such as at least six delivery fibres, such as at least 8 delivery fibres, such as at least ten delivery fibres. The number of fibres being a compromise between maximising the number of fibres in order to obtain a homogenous light delivery to the tissue, while on the other hand to maintain sufficient effect in each fibre.

When a bundle of fibres are arranged in the system, the bundle may comprise at least 5 fibres, such as at least 10 fibres, such as a bundle of at least 20 fibres, such as a bundle of at least 30 fibres.

The distal end of the delivery fibre may be adapted for insertion, such as being processed to optimize insertion, such as to have a substantially conical shap, being tapered, or as to have a lens mounted (GRIN lens).

In particular in a system comprising one optical fibre in serial coupling to several delivery fibres the system preferably comprises means for coupling comprising first coupling means for coupling the at least substantially elliptic beam into at least one optical fibre, and second coupling means for coupling the beam from the at least one optical fibre into individual delivery fibres capable of delivering laser light to the biological tissue.

Accordingly, the system preferably provides means for splitting the beam from the optical fibre into at least two individual delivery fibres, such as a system wherein the means for splitting the beam comprises direction means comprising at least one beam splitter capable of splitting the beam into at least two beams, and second coupling means for coupling the at least two beams into individual delivery fibres.

The system preferably comprises direction means comprising a connector for receiving the light beam from the optical fibre before entering the beam splitter.

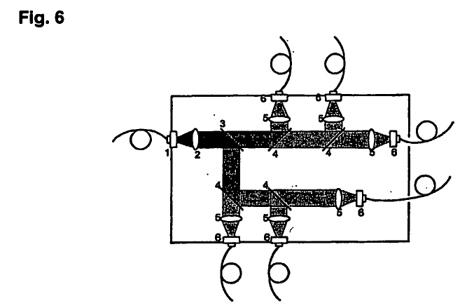

The beam is split into at least two individual beams corresponding to delivery to at least two individual delivery fibres. Therefore the direction means preferably comprises at least two beam splitters for splitting the beam into at least three beams, such as direction means comprising at least five beam splitters for splitting the beam into six beams. In a preferred configuration the direction means is a setup as shown in FIG. 6.

One of the greatest difficulties when applying the laser light to the tissue, is to correctly calculate and control the delivered light dose to the tissue. An even distribution is preferred, and it is of importance that no area of the tumor tissue is left intreated. Optical dosimetry is a not well developed technology, due to little knowledge of absorption and distribution properties of the tissue. The effect of the treatment depends on the amount of light delivered to the cells. Since the light amount is determined by the delivered dose, the absorption and the scatter, a need for measuring the amount of light in situ in the tissue is present.

It is accordingly of interest to use at least one delivery fibre as a detector monitoring the laser light emitted. The fibre used as detector preferably comprises a light blocking means so that the light delivery of said fibre may be shut off when it functions as detector.

The detector preferably detects at least one of the parameters of relevance for the dosimetry of the treatment, the treatment being photodynamic therapy or laser-induced thermo-therapy. The parameters of interest to detect would be the treatment light distribution and fluence rate, the sensitizer concentration and consumption, oxygen content, and tissue temperature. All of these measurements could be performed by optical means by using the proposed detection method. These measurements may be used to infer the tissue parameters of interest for the treatment progression, e.g., optical and thermal properties.

The temperature could be measured by the detecting the fluorescence from specially prepared fibre tips inserted into the tissue. These fibre tips fluoresces due to rare-earth metal-doped crystals incorporated in the tip.

All of these parameters could be measured by using temporally pulsed light sources, intensity or frequency modulation of the light source as well as continuous wave sources.

For the various types of treatments, different temporal of frequency modulation schemes may be advantageous in combination with the proposed laser system, the light delivery and detection system.

In a preferred embodiment all the fibres are capable of acting as delivery fibres and detectors. Thereby it is possible to sequentially measure the light flow from one fibre to the other fibres and the dose may be modelled with a tomographic method. During the treatment measurements may be obtained several times to calculate the exact amount of light to be delivered. Preferably also, the amount of light may be calculated per fibre, to deliver different amounts of light in the individual fibres to correspond to the treatment effect.

It is preferred that the delivery of light and monitoring is carried out in the same procedure and fibres. Accordingly, it is preferred that a detector is coupled to at least one of the delivery fibres, and even more preferred that a light blocking means may be coupled to at least one of the delivery fibres, so that the light delivery is shut off when the monitoring is conducted. Preferably, at least one fibre, more preferably all fibres used comprises both a detector and a light blocking means is coupled to the delivery fibre. As discussed above, the fibres are preferably used sequentially as detectors.

Using laser excitation in the appropriate wavelength range, characteristic fluorescence from the photosentisizer is generated, whereby photo-activated fluorescence monitoring of malignant tumors is enabled. The monitoring procedure is limited to relatively low light and thus signal levels and the laser system should preferably operate in pulsed mode to reduce the influence of the ambient background light.

The distal end of said delivery fibres are arranged in contact with the biological tissue. In case of superficial tissue the term in contact with relates to merely applying the fibres to the tissue, whereas for more profoundly located tissue, the delivery fibres may be inserted into the tissue, of course a combination of contacting and inserting fibres may be used. The delivery of light to the tissue is preferably conducted sequentially with monitoring phases interposed between delivery phases to monitor the delivery throughout the whole delivery period. The delivery fibres are normally in contact with the tissue for a period of time, said period being in the range of from 2 minutes to 180 minutes depending on the effect delivered and the effect necessary, taken into account that tissue surrounding the tissue to be treated should preferably not receive any laser light, or only very small amounts of laser light. The period is often from 10 to 30 minutes.

The system according to the invention is particular suitable for treatment of a condition or disease wherein said condition or disease is susceptible to thermo-treatment and/or photodynamic treatment.

Solid, deep lying tumors may be treated with lasers, either using the process of PDT or by just due to the absorbed light heat the tissue sufficiently to kill it—thermo therapy. However, in this case the light must be delivered to the tumor, inside as well as close to the tumor boundaries. This procedure is denoted interstitial laser treatment. In order to achieve as even treatment as possible, it is favourable to utilize several sources at different positions. The tumor may be a malignant or a premalignant tumor.

Photodynamic treatment is based on administration of a photo-sentisizer to the individual to be treated and then emission of light to the relevant tissue. The treatment relies on the coexistence of light, oxygen and a photosensitive component, a photosentisizer. The photosentisizer has the ability to accumulate to a higher degree in these types of diseased tissue than in the surrounding healthy tissue.

The ability of accumulation may be accomplished by several mechanisms. The photosensitizer may be coupled to specific ligands reactive with the target, such as receptor-specific ligands, or immunogolbulins or fragments thereof as described in WO 00/41726, which is hereby incorporated by reference. The photosensitizer may also be activatable only in the target tissue, for example by enzyme reactions. The treatment light excites the photosensitizer and the excessive energy from this process leads to a photochemical formation of the aggressive and highly reactive singlet molecular oxygen (¹Δ_{g}). The singlet oxygen acts oxidative to its surroundings, whereby causing severe damage to the cancerous cells, leading to selective cell necrosis of the diseased tissue. Photo-sentisizers may be porfyrins, ftalocyanins, etc, capable of accumulating in the tissue to be treated. The photosensitizer is excited with the light. The photosensitizer is preferably selected from Haematoporphyrin IX, Photofrin, protoporfyrin, Haematoporphyrin derivative, mono-aspartyl chlorin, benzoporphyrin derivative monoacid ring A, tetra-sulphonated aluminium phtalocyanine. In a preferred embodiment the prophotosensitizer ALA (6-aminolevulinic acid) is administered. ALA is then converted to Haematoporphyrin IX accumulating in for example tumor tissue, see also table 1 below with respect to selected wavelength for each photosensitizer.

TABLE 1

| | PDT | | | Fluorescence | | | |
|---|---|---|---|---|---|---|---|
| | φ_{Δ} | λ_{PDT} | α | λ_{exc} | λₑₘ | τ | φ_{F} |
| Sensitizer | (%)^{a} | (nm)^{a} | (cm² J⁻¹)^{a} | (nm)^{b} | (nm)^{b} | (ns)^{b} | (%)^{b} |
| HP | 73 | 630 | 30 | 405 | 610 | 13.5 | 9 |
| HpD | | | | 400 | 610 | 15.5, 2.5 | 2-7 |
| PpIX | 56 | 630 | 110 | | | | |
| PF | 89 | 630 | 30 | | | | <10^{c} |
| Chl-e6 | 64 | 662 | 220 | 410* | 664* | 3.7 | |
| BPD-MA | 84 | 687 | 320 | 400 | 690 | 5.5 | 10-20^{c} |
| AlS₄Pc | 38 | 673 | 760 | 350 | 675 | 5.3 | 30-50^{c} |

^{a}Ref. (120); ^{b}Ref. (121); Ref. (122)
*MACE

Table 1 Photophysical and photochemical properties of some photosensitizers. Φ_{Δ}: singlet oxygen yield; λ_{PDT}: wavelength used for PDT; α: singlet oxygen generation rate at λ_{PDT}; λ_{exc}; λₑₘ: main fluroscence excitation and emission wavelengths, respectively; Φ_{F}: fluroscence quantum yield, Hp: haematoporphyrin; HpD: haematoporphyrin derivative; PpIX: protoporphyrin IX; PF: Photofrin; ChI-e6: chlorin e₆; MACE: mono-aspartyl chlorin e₆; BPD-MA: benzoporphyrin derivative monoacid ring A; AIS₄Pc: tetra-sulphonated aluminium phthalocyanine.

The photosensitizer in question may be administered to the individual in an amount sufficient to obtain the relevant concentration in the target tissue. The route of administration may be any suitable route, such as orally, or by injection, such as but not limited to intravenous, subcutaneous, intramusular or intraperitoneal. Also, enterally or topically administration forms are possible.

Tumors may also be treated by thermo-treatment, leading to hyperthermia, which is also induced by for example laser light. Tumor cells normally are more sensible to temperature differences. Increase of temperature may lead to coagulation and subsequently necroses of the tumor tissue.

The method of treatment includes identification of the tissue volume to be treated. The identification may be conducted for example by visual inspection, biopsies and/or ultrasound.

The at least two delivery fibres connected to a system as defined above are arranged in contact with at least a part of said tissue volume and the laser light is emitted through said fibres, optionally monitored as described above. In case of photodynamic treatment the emission of light is preceded by administration of a photosensitizer or a precursor of a photosensitizer.

As discussed above the condition or disease treated according to the invention may be a tumor, such as a malignant tumor.

In another aspect of the invention, the system and method may be used for diagnostic purposes, such as a system for diagnosing a condition or disease, such as a tumor.

The method of diagnosing a condition or disease relating to a tissue volume in an individual comprises, arranging at least two delivery fibres connected to a system as defined above in contact with tissue suspected to comprise at least a part of said tissue volume, emitting laser light through said delivery fibres to the tissue volume, detecting a signal in at least one of the other delivery fibres, and correlating said signal to the presence or absence of said condition or disease.

The signal detected is normally a fluorescent signal. The signal may be enhanced by administering a tumor marker to said individual before emitting laser light. In particular for diagnostic purposes the arrangement of fibres may be a bundle of fibres as defined above wherein the distal ends are arranged in contact with the tissue.

Furthermore, for diagnostic purposes it is an advantage that the laser light emitted is frequency modulated. In optical communication system, light signals are generated by modulation of light emitted from a semiconductor laser by modulation of the current supplied to the laser. However, the wavelength of light emitted by a free running semiconductor laser varies as a function of the current supplied to the laser and thus, modulation of the supply current causes generation of light frequency chirp, typically corresponding to 1 nm wavelength chirp, which lowers the available capacity of communication channels of the communication system. It is an important advantage of the invention that light frequency chirp caused by modulation of current supplied to the first laser is substantially eliminated.

### EXAMPLE

The example shows a setup for delivery of laser light to a biological tissue to an individual wherein said individual has received ALA as a precursor for a photosensitizer.

Experiments

The setup scheme can be explained from FIG. 2 as follows. The output from a multimode diode laser consists of a superposition of lateral broad area modes. Each of the modes constitutes a double lobed intensity profile in the far field. The different modes are distinguished by different frequencies and radiation angles. Mode number m exhibits two symmetrical lobes in the far-field plane, with peak intensities radiated at angles ±θₘ, with respect to the normal of the emitting aperture of the laser, i.e. the z-axis. The fundamental mode is radiated at the angle θ=0. By the insertion of a spatial filter one can choose a single lobe of the double lobed profile to pass. The linear spatial filter is placed in the far-field plane defined by the collimating lenses L1 and L2. The lobe transmitted through the spatial filter is reflected back into the laser medium by the external reflector. This causes the laser to prefer lasing in one spatial mode. The resulting single-mode radiation is strongly asymmetric with a dominant lobe, referred to as the output lobe, and a smaller lobe, referred to as the injection lobe. As shown in FIG. 2 the smaller lobe runs between the diode laser and the external reflector, locking the diode laser in a single spatial mode operation. The experimental setup shown in FIG. 4 illustrates a AlGaInP broad area single-stripe laser-diode (HPD1 302-TO3-TEC) implemented in the feedback system. A silver coated mirror acts as spatial filter and feedback component. This component is placed in the far field defined by L1 and L2.

The dimensions of the emitting aperture of the BAL are 1 μm in the transverse (the high coherence axis—y) and 100 μm in the lateral (the low coherence axis—x) direction. The laser is linearly polarized in the y-direction, which is also the high coherence axis of the laser. The maximum output power of the freely running laser is 200 mW. This power is obtained at a drive current of 550 mA≅1.7×Iₜₕ, where Iₜₕ denotes the threshold current of the BAL, Iₜₕ=330 mA for the freely running laser. The bandwidth (FWHM) of the BAL is 0.7 nm. L1 and L2 constitute a collimating lens pair with focal lengths of 4.5 mm (aspherical) and 40 mm (cylindrical) respectively, while W is a wedge extracting two reflections to beam diagnostics. E is an optional frequency selective element (e.g. a Fabry-Perot etalon) that, when inserted correctly, forces the BAL to oscillate in a single frequency. The etalon may be omitted, since the absorption band of the photosentisizer is relatively large as compared with the bandwidth of the laser. MF denotes the external feedback mirror and the spatial filter, which are placed in the far field plane defined by L1 and L2. The scheme forces the laser to emit single-mode radiation where one of the lobes (the output lobe) is amplified. The high-power lobe is extracted from the system by the mirror M, as shown in FIG. 2.

The output is collimated in the high coherence axis, but is slightly diverging in the low coherence axis. In order to couple the output into an optical fibre with a core diameter of 50 μm, the beam is expanded in the lateral direction to obtain an approximately circular shape before the beam is focused into the fibre. The beam expanding system consists of two cylindrical lenses of focal lengths 5 mm (L3) and 40 mm (L4), respectively (See FIG. 4). The beam is coupled into the optical fibre using an achromat (L5) with a focal length of 40 mm. The length of the setup without the beam splitting, delivery and monitoring unit is 30 cm and it was built on a lightweight honeycomb breadboard of dimensions 45×60 cm² for portability. The 50 m core-diameter fibre is coupled into the beamsplitting, delivery and monitoring unit as depicted in FIG. 4.

Results

The lateral far-field pattern, i.e. the intensity distribution, was measured as a function of radiation angle. The distributions were measured in the beam path of one of the reflections from the wedge in FIG. 4. They were measured in the far-field plane defined by L1 and L2, see FIG. 5. The beam quality factor M² is enhanced from an M²=8 when the laser is freely running to M²=1.6 when the feedback scheme is applied at a drive current of 1.3×Iₜₕ At I=1.7×Iₜₕ the M² is improved from 9 to 1.9.

The beam was expanded to a substantially circular beam of approximately 8×8 mm². The fibre is a 50 μm core diameter glass fibre with a numerical aperture of 0.25. Approximately 80 percent of the output power from the laser system is coupled through the fibre.

1. A system for emitting laser light to a biological tissue comprising:
a laser light system having low spatial coherence, a first laser for emission of a first power light beam,
means for improving the laser light system from low coherence to high coherence, to obtain a system capable of producing an output beam consisting of high temporal and/or high spatial coherence, wherein said means comprises
means for external feedback for emission of a second light beam in response to light incident upon it and being positioned in relation to the first laser so that, during emission of the first light beam, the device is illuminated by a first part of the first light beam and the second light beam is injected into the first laser, the means for external feedback and the first laser defining an external cavity there between, and/or
a spatial filter,
focusing means for focusing said output beam, and
means for coupling said beam into at least one delivery fibre capable of delivering the laser light to the biological tissue, and wherein a detector is coupled to at least one of the delivery fibres for monitoring the laser light emitted.

2. The system according to claim 1, wherein the means for coupling comprises first coupling means for coupling beam into at least one optical fibre, and second coupling means for coupling the beam from the at least one optical fibre into individual delivery fibres capable of delivering laser light to the biological tissue.

3. The system according to claim 2, further comprising means for splitting the beam from the optical fibre into at least two individual delivery fibres.

4. The system according to claim 3, wherein the means for splitting the beam comprises direction means comprising at least one beam splitter capable of splitting the beam into at least two beams, and second coupling means for coupling the at least two beams into individual delivery fibres.

5. The system according to claim 1, wherein the at least one delivery fibre is a bundle of fibres, such as a bundle of at least 10 fibres, such as a bundle of at least 20 fibres, such as a bundle of at least 30 fibres.

6. The system according to claim 1, wherein the laser light system comprises a member selected from the group consisting of: broad area laser, laser diode array, laser bar, stacked array, laser diode, and combinations thereof.

7. The system according to claim 6, wherein the means for external feedback comprises phase conjugating means, comprising a member selected from the group consisting of: a mirror, a holographic element, a grating, and combinations thereof.

8. The system according to claim 1, wherein the means for external feedback comprises an etalon.

9. The system according to claim 1, wherein the laser is capable of producing a beam having a wavelength in the range of from 250 nm to 1600 nm, such as from 300 nm to 1400 nm, such as from 400 nm to 800 nm.

10. The system according to claim 1, wherein the laser light system comprises means for non-linear generation of other frequencies.

11. The system according to claim 10, wherein the means for non-linear generation of other frequencies is an intracavity means.

12. The system according to claim 10, wherein the other frequencies has a wavelength in the range of from 250 nm to 1600 nm.

13. The system according to claim 1, comprising a reflecting means for directing the output beam towards the focusing means.

14. The system according to claim 1, wherein the focusing means comprises means for expanding the output beam in at least one direction.

15. The system according to claim 1, wherein the focusing means comprises means for expanding the output beam into a substantially circular beam.

16. The system according to claim 15, wherein the focusing means comprises means for expanding the output beam at least 4 times in at least one direction.

17. The system according to claim 14, wherein the coupling means comprises means having a numerical aperture adapted to the diameter of the at least one fibre.

18. The system according to claim 1, wherein the first coupling means comprises a lens, such as an achromat or a triplet.

19. The system according to claim 4, wherein the direction means comprises a connector for receiving the light beam from the optical fibre before entering the beam splitter.

20. The system according to claim 4, wherein the direction means comprises at least two beam splitters for splitting the beam into at least three beams.

21. The system according to claim 4, wherein the direction means comprises at least five beam splitters for splitting the beam into six beams.

22. The system according to claim 1, wherein a light blocking means is coupled to at least one of the delivery fibres.

23. The system according to claim 1, wherein a detector and a light blocking means is coupled to at least one of said at least one delivery fibre.

24. The system according to claim 1, wherein diameter of the core of the delivery fibre is in the range of from 5 μm.

25. The system according to claim 1, wherein the at least one delivery fibre has been processed to optimize insertion.

26. A method for emitting laser light to a biological tissue comprising:
arranging a laser light system having low spatial coherence,
improving the laser light system from low coherence to high coherence, to obtain a system capable of producing an output beam consisting of high temporal and/or high spatial coherence,
focusing said output beam,
coupling said beam into at least one delivery fibre having a proximal end and a distal end, and
arranging the distal end of said delivery fibres in contact with the biological tissue.

27. The method according to claim 26, wherein the output beam is coupled into at least one optical fibre, and the beam from the at least one optical fibre is coupled into individual delivery fibres capable of delivering laser light to the biological tissue.

28. The method according to claim 26, wherein the beam from the optical fibre is split into at least two individual delivery fibres.

29. The method according to claim 28, wherein the beam is split by means of splitting means comprising direction means comprising at least one beam splitter capable of splitting the beam into at least two beams, and second coupling means for coupling the at least two beams into individual delivery fibres.

30. The method according to claim 26, wherein the at least one delivery fibre is a bundle of fibres.

31. The method according to claim 26, wherein the laser light system comprises a member selected from the group consisting of: a broad area laser, a laser diode array, a laser bar, a stacked array, of a laser diode, and combinations thereof.

32. The method according to claim 26, wherein the laser light system comprises means for external feedback.

33. The method according to claim 26, wherein the means for external feedback comprises phase conjugating means, such as a mirror, a holographic element or a grating.

34. The method according to claim 33, wherein the means for external feedback comprises a spatial filter.

35. The method according to claim 33, wherein the means for external feedback comprises an etalon.

36. The method according to claim 26, wherein the laser is capable of producing beam having a wavelength in the range of from 250 nm to 1600 nm.

37. The method according to claim 26, wherein the laser light system comprises means for non-linear generation of other frequencies.

38. The method according to claim 37, wherein the means for non-linear generation of other frequencies is an intracavity means.

39. The method according to claim 37, wherein the other frequencies have a wavelength in the range of from 250 nm to 1600 nm.

40. The method according to claim 26, comprising a reflecting means for directing the output beam towards the focusing means.

41. The method according to claim 26, wherein the focusing means comprises means for expanding the output beam in at least one direction.

42. The method according to claim 26, wherein the focusing means comprises means for expanding the output beam into a substantially circular beam.

43. The method according to claim 42, wherein the focusing means comprises means for expanding the output beam at least 4 times in at least one direction.

44. The method according to claim 42 or 13, wherein the coupling means comprises means having a numerical aperture adapted to the diameter or thickness of the core of the at least one fibre.

45. The method according to claim 26, wherein the first coupling means comprises a lens.

46. The method according to claim 30, wherein the direction means comprises a connector for receiving the light beam from the optical fibre before entering the beam splitter.

47. The method according to claim 30, wherein the direction means comprises at least two beam splitters for splitting the beam into at least three beams.

48. The method according to claim 30, wherein the direction means comprises at least five beam splitters for splitting the beam into six beams.

49. The method according to claim 26, wherein the delivery fibre is capable of functioning as a detector.

50. The method according to claim 26, wherein a light blocking means may be coupled to at least one of the delivery fibres.

51. The method according to claim 26, wherein the delivery fibre is capable of functioning as a detector, and a light blocking means is coupled to said delivery fibre.

52. The method according to claim 49, wherein the detector is detecting at least one of: fluorescent radiation from the tissue, temperature of the tissue, and light flow is detected from the tissue.

53. The method according to claim 49, wherein the fibres are used sequentially as a detector.

54. The method according to claim 26, wherein diameter of the core of the delivery fibre is in the range of from 5 μm to 500 μm.

55. The method according to claim 26, wherein the delivery fibres are in contact with the tissue for a period of time, said period being in the range of from 2 minutes to 180 minutes.

56. The method according to claim 26, wherein at least one of said at least one delivery fibre is inserted into the biological tissue.

57. The method according to claim 56, wherein the at least one delivery fibre has been processed to optimize insertion.

58. (Canceled)

59. (Canceled)

60. (Canceled)

61. A method of treating a condition or disease relating to a tissue volume in an individual comprising,
identifying the tissue volume,
arranging at least one delivery fibre connected to a system as defined in claim 1 in contact with at least a part of said tissue volume,
emitting laser light through said delivery fibre to the tissue volume.

62. The method according to claim 61, wherein the laser light emitted by the delivery fibre is monitored.

63. The method according to claim 62, wherein at least one of said at least one delivery fibre is capable of functioning as a detector monitoring the laser light emitted.

64. The method according to claim 61, wherein a light blocking means is coupled to at least one of said at least one delivery fibre.

65. The method according to claim 62, wherein a light blocking means is coupled to said at least one delivery fibre which is capable of functioning as a detector.

66. The method according to claim 63, wherein the detector is detecting one or more of fluorescent radiation from the tissue, temperature of the tissue and light flow from the tissue.

67. The method according to any of the claim 63, wherein said at least one fibre is used sequentially as a detector.

68. The method according to claim 61, wherein the treatment is a thermotreatment.

69. The method according to claim 61, wherein the condition or disease is a tumor.

70. The method according to claim 61, wherein a photosensitizer or a precursor to a photosensitizer is administered to said individual before emitting laser light.

71. The method according to claim 70, wherein the photosensitizer is selected from Haematoporphyrin IX, Photofrin, protoporfyrin, Haematoporphyrin derivative, mono-aspartyl chlorin, benzoporphyrin derivative monoacid ring A, tetra-sulphonated aluminium phtalocyanine.

72. (Canceled)

73. (Canceled)

74. A method of diagnosing a condition or disease relating to a tissue volume in an individual comprising,
arranging at least two delivery fibres connected to a system as defined in any of the claims 125 or by the method as defined in claim 1, in contact with tissue suspected to comprise at least a part of said tissue volume,
emitting laser light through at least one of said delivery fibres to the tissue volume,
detecting a signal in at least one of the other delivery fibres, and
correlating said signal to the presence or absence of said condition or disease.

75. The method according to claim 74, wherein said condition or disease is a tumor.

76. The method according to claim 75, wherein said signal is a fluorescent signal.

77. The method according to claim 75, wherein a tumor marker is administered to said individual before emitting laser light.

78. The method according to claim 74, wherein the delivery fibres are a bundle of fibres having movable distal ends.

79. The method according to claim 74, wherein the laser light emitted is frequency modulated.
